(19) United States
(12) Patent Application Publication
(10) Pub. No.: US 2020/0334229 A1
(43) Pub. Date: Oct. 22, 2020

(54) MEDICAL DEVICE BLOCKCHAIN EXCHANGE

(71) Applicant: Hill-Rom Services, Inc., Batesville, IN (US)

(72) Inventors: Patrick D. HARRISON, St. Johns, FL (US); William B. RICHARD, Jamestown, RI (US)

(21) Appl. No.: 16/843,121

(22) Filed: Apr. 8, 2020

Related U.S. Application Data
(60) Provisional application No. 62/835,275, filed on Apr. 17, 2019.

(51) Int. Cl. G06F 16/23; G16H 40/67; G06Q 10/10; G06F 21/62; G06F 21/60; A61G 7/018

(52) U.S. Cl. CPC G06F 16/2379; G16H 40/67; G06Q 10/10; G16H 40/40; G06F 21/6245; G06F 21/602; A61G 7/018; G06F 16/2365

(57) ABSTRACT

Devices, systems, and methods for blockchain data management for interaction of medical devices include distributed ledger of medical device information. Medical devices can form nodes of a medical device network for maintaining a device level interaction ledger. Medical devices can communicate with a network to manage access to the medical device interaction data.

### CROSS-REFERENCE TO RELATED APPLICATIONS

This U.S. Non-Provisional patent application claims the benefit of priority of U.S. Provisional Application No. 62/835,275, filed on Apr. 17, 2019, the disclosure of which is incorporated by reference in its entirety, including but without limitation, those portions related to medical devices and/or communications.

### BACKGROUND

The present disclosure relates to devices, systems, and methods for medical device communication. More specifically, the present disclosure relates to devices, systems, and methods for medical device communications.

Medical device communication is increasing rapidly. Managing, accessing, and/or securing such medical device communication is becoming increasingly challenging. Moreover, the resources required for adequate operation of communications between and concerning medical devices is increasing. Appropriate management and access to medical devices communications can provide enhanced patient outcomes, while providing additional sources for optimizing patient care.

### SUMMARY

The present application discloses one or more of the features recited in the appended claims and/or the following features which, alone or in any combination, may comprise patentable subject matter.

According to an aspect of the present disclosure, a patient bed for blockchain exchange of process of care information, including a bed frame for supporting a patient above the floor, and a blockchain exchange system for supporting a distributed ledger of interactions between the patient bed and other medical devices. The blockchain exchange system may be secured with the frame and may include a processor, at least one memory storage, and communication circuitry, wherein the processor is configured to execute instructions stored on the at least one memory storage to validate and record interactions between the patient bed and the other medical devices, wherein each valid interaction is linked with at least one previous valid interaction.

In some embodiments, valid interactions may be grouped together into at least one block of interactions. Each block of interactions may be linked to at least one preceding block of interactions. Each block may include a cryptographic arrangement of its grouped valid interactions.

In some embodiments, the blockchain exchange system may be configured to validate an interaction between the patient bed and another medical device by identification of the other medical device. Identification of the other medical device may include receiving an indication of an identification code from the other medical device. Identification of the other medical device may include exchanging information with the other medical device. Identification of the other medical device may include determining that the medical device is within a threshold proximity of the patient bed.

In some embodiments, validation of interactions between the patient bed and other medical devices may include communicating identification of the other medical device to at least one other device configured to validate and record interactions between the patient bed and the other devices. The blockchain exchange system may be configured to communicate interactions between the patient bed and other medical devices to at least one other device configured to validate and record interactions, and to receive indication of at least one interaction as a block linked with at least one previous interaction. In some embodiments, the blockchain exchange system may be configured to record the block linked with at least one previous interaction.

According to another aspect of the present disclosure, a medical device communication system for blockchain exchange, may include a patient bed for supporting a patient above the floor, the patient bed having a blockchain exchange system including a processor executing instructions stored on at least one memory storage and communication circuitry for communication with other medical devices, a remote server arranged in communication with the patient bed, and a network of medical device nodes each including a blockchain exchange system including a processor executing instructions stored on at least one memory storage and communication circuitry for communication with other medical device nodes of the network. The blockchain exchange system of at least one of the patient bed and one of the medical device nodes may be configured to validate interactions between the patient bed and other medical devices, and each of the blockchain exchange systems is configured to record valid interactions linked with at least one previous valid interaction between the patient bed and other medical devices.

In some embodiments, the blockchain exchange system of the patient bed may validate interactions between the patient bed and other medical devices. The blockchain exchange systems of the patient bed and at least one of the medical device nodes may compete to determine which will validate an interaction between the patient bed and another medical device. The successful one of the blockchain exchange systems of the patient bed and at least one of the medical device nodes competing to determine which will validate an interaction between the patient bed and another medical device, may send a validation signal indicating the valid interaction to the other medical device nodes of the network.

In some embodiments, each of the blockchain exchange systems of the patient bed and the medical device nodes may maintain an identical ledger of valid interactions. The interactions between the patient bed and other medical devices may include an exchange of information between the patient bed and the other medical device. The exchange of information may be process-of-care information. The exchange of information may not include Protected Health Information.

The remote server may be configured to perform agreement validation to permit a medical device to join the network of medical device nodes. The agreement validation may be a blockchain exchange. The agreement validation may include smart contract formation hosted on the remote server. Smart contract formation may include authorization for access to recorded valid interactions between patient bed and other medical devices. The recorded valid interactions may be encrypted.

According to another aspect of the present disclosure, a medical device communication system for blockchain exchange, may include a patient bed for supporting a patient above the floor, the patient bed including a blockchain exchange system including a processor executing instructions stored on at least one memory storage and communication circuitry for communication with other medical devices. The blockchain exchange system may be configured to validate and record interactions between the patient bed and other medical devices, wherein each valid interaction is linked with at least one previous valid interaction. The communication system may include a remote server arranged in communication with the patient bed, and a network of medical devices each including a processor executing instructions stored on at least one memory storage and communication circuitry for communication with other medical devices of the network. At least one of the medical devices of the network may form a network node configured to validate and record interactions between the patient bed and other medical devices.

In some embodiments, the patient bed and at least one of the medical devices may forming the network node may compete to determine which will validate an interaction between the patient bed and another medical device. The successful one of the patient bed and at least one of the medical device forming the network node competing to determine which will validate an interaction between the patient bed and another medical device, may send a validation signal indicating the valid interaction to the other medical devices of the network. The patient bed and the medical devices of the network may maintain an identical ledger of valid interactions.

In some embodiments, the interactions between the patient bed and other medical devices may include an exchange of information between the patient bed and the other medical device. The exchange of information may be process-of-care information. The exchange of information may not include Protected Health Information.

In some embodiments, the remote server may be configured to perform agreement validation to permit a medical device to join the network of medical devices. The agreement validation may be a blockchain exchange. The agreement validation may include smart contract formation hosted on the remote server. Smart contract formation may include authorization for access to recorded valid interactions between patient bed and other medical devices. The recorded valid interactions may be encrypted.

According to another aspect of the present disclosure, a medical information communication system for blockchain exchange, may include a patient bed for supporting a patient, the patient bed including a device-level blockchain exchange system for recording valid interactions of the patient bed with other medical devices, the device-level blockchain exchange system including a processor executing instructions stored on at least one memory storage and communication circuitry for communication with other medical devices, and a remote server arranged in communication with the patient bed and including a network-level blockchain exchange system for preforming agreement validation. The blockchain exchange system may be configured to receive requests for access to recorded valid interactions of the patient bed with other medical devices, to perform agreement validation for each request, to authorize access to recorded valid interactions based on the agreement validation for each request, and to record successful agreement validations linked with at least one previous recorded successful agreement validation.

In some embodiments, the remote server may be configured to receive requests for access from medical devices not within a network of medical devices authorized for access to recorded valid interactions. The network of medical devices may include at least one medical device forming a network node and configured for validating interactions of the patient bed with other medical devices. The network of medical devices may include at least one medical device configured to record valid interactions of the patient bed with other medical devices.

In some embodiments, configuration to perform agreement validation may include confirming identity of a source device of the request, building an agreement profile, and ratifying the agreement profile with a source device of the request. Responsive to successful ratification of the agreement profile, the remote server may provide an access key to the source device to access recorded valid interactions of the patient bed with other medical devices. The access key may be formed to limit access to record valid interactions of the patient bed with certain other medical devices.

In some embodiments, confirming identity of a source device of the request includes reviewing an identification code provided by the source device. Recorded valid transactions may include at least one of configuration, identification number, device type, device location, and a communication certificate of the other medical device interacting with the patient bed.

Additional features, which alone or in combination with any other feature(s), including those listed above and those listed in the claims, may comprise patentable subject matter and will become apparent to those skilled in the art upon consideration of the following detailed description of illustrative embodiments exemplifying the best mode of carrying out the invention as presently perceived.

### BRIEF DESCRIPTION OF THE DRAWINGS

The detailed description particularly refers to the accompanying figures in which:

FIG. 1 is a perspective view of a patient bed within a room of a care facility including a blockchain exchange system communicating with other medical devices to maintain a distributed ledger of device interactions, and showing that the patient bed includes a graphical user interface for access to bed information;

FIG. 2 is a diagrammatic view of the blockchain exchange system of the patient bed of FIG. 1 in communication with a network of medical device nodes;

FIG. 3 is a flow diagram of a device interaction, such as interaction of the patient bed of FIG. 1 with another medical device, showing that a validation can include requesting access to a remote server, validating agreement for access, forming a recording the validated agreement within a blockchain, and communicating the successful agreement validation to the medicals devices of interaction;

FIG. 4 is diagrammatic view of block formation of a blockchain illustrating that new valid interactions are recorded within blocks connected with previous blocks of the chain;

FIG. 5 is a diagrammatic view of a series of layers of communication including the blockchain agreement validation of FIG. 3;

FIG. 6 is a flow diagram of blockchain operations including agreement validation of FIGS. 3 and 5 and mining of blockchain information in remote servers;

FIG. 7 is a flow diagram of blockchain operations, similar to FIG. 6, including agreement validation and access of blockchain information by a medical device;

FIG. 8 is screenshot of the graphical user interface of the patient bed of FIG. 1, showing that an exchange registry of confirmed medical devices and an interaction ledger of valid interactions, each of which can be viewed by an authorized user;

FIG. 9 is a screenshot of the graphical user interface of the patient bed of FIG. 1, showing that an authorization screen can be presented responsive to a request for access to the interaction ledger, and showing that buttons are presented for user selection to accept or deny the authorization request; and

FIG. 10 is a screen shot of a the graphical user interface of the patient bed of FIG. 1, showing that an authorization request code can be prompted in response to the user selection to accept the authorization request of FIG. 9.

### DETAILED DESCRIPTION

For the purposes of promoting an understanding of the principles of the disclosure, reference will now be made to a number of illustrative embodiments illustrated in the drawings and specific language will be used to describe the same.

The rapid expansion of communication in the area of patient care presents challenges of safety and privacy, but also affords the opportunity to provide new and/or repurposed value from the data available. Blockchain architecture can offer secure platforms for information exchange. Maintaining and accessing information regarding the interaction of medical devices using blockchain architecture, alone or in collaboration with Internet-of-Things (IoT) and/or artificial intelligence, can enable secure information exchange and management well-suited to the objectives of patient care.

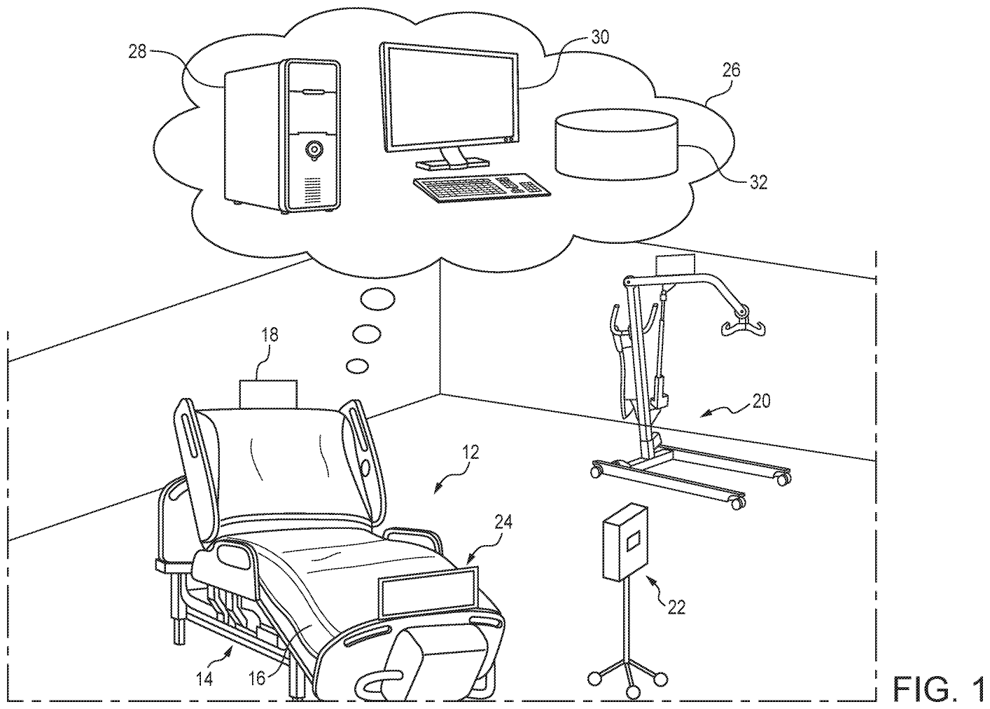

Referring to the illustrative embodiment as shown in FIG. 1, a patient bed 12 is shown located within a room of a care facility, such as a hospital. The patient bed 12 includes a frame 14 which supports a mattress 16 for supporting a patient above the floor. The patient bed 12 is illustratively embodied as configurable patient support having a variety of adjustable features including mattress height, torso tilt, and leg tilt. The patient bed 12 includes a blockchain exchange system 18 for supporting a distributed ledger of interactions between medical devices.

The blockchain exchange system 18 is illustratively arranged to support communication with other medical devices, such as patient lift 20 and/or intravenous (IV) pump 22. The blockchain exchange system 18 is adapted to record interactions with other medical devices to support a ledger of interaction activity between medical devices. Interaction between medical devices can include proximity between the medical devices within a threshold distance. For example, when the IV pump 22 is arranged within a threshold distance from the patient bed 12, an interaction may be defined by the proximity event in order to track the nature of the patients having occupied the patient bed 12 against the exposure of the IV pump 22. More specifically, by tracking the proximity events as interactions between the patient bed 12 and the IV pump 22, the exposure of the IV pump 22 to patient conditions, such as diseases, can be tracked according to the correspondence of the patients with the patient bed 12. Device interactions may include other manners of interaction such as direct and/or indirect communications and/or physical connection between the devices; and/or application of the devices to common patients, by common caregivers, and/or in common procedure types.

The patient bed 12 illustratively includes a graphical user interface (GUI) 24. The GUI 24 is illustratively embodied as a touch screen for user operation to view and interact with the blockchain exchange system 18 information, such as the medical device interaction ledger. As discussed in additional detail herein, the GUI 24 can present information regarding medical device interaction for user consideration and operation.

The patient bed 12 is illustratively arranged in communication with network 26. The network 26, embodied as a hospital network, includes and/or communicates with devices remote from the patient bed 12 such as servers 28, user interfaces 30, and/or storage devices 32. The network 26 may be arranged in communication with other networks, such as the Internet, and the remote devices may be located remote from the care facility. As discussed in additional detail herein, the network 26 can permit validation of agreements to provide new medical devices with access the medical device interaction ledger, and/or may enable access for third party services to the interaction ledger.

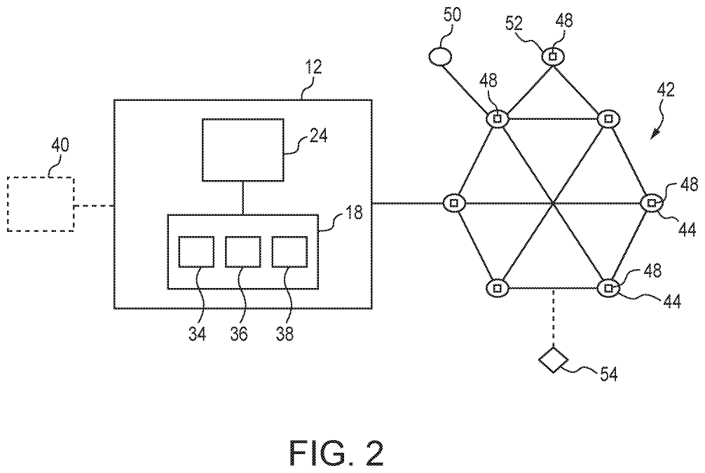

Referring now to FIG. 2, the blockchain exchange system 18 illustratively includes a processor 34, memory storage 36, and communication circuitry 38 for communication with other medical devices. In some embodiments, the patient bed 12 may include other processors, memory storages, and/or communication circuitry for operations other than blockchain-related operations, and/or may have partly or wholly shared hardware and/or software components with the blockchain exchange system 18. For example, the patient bed 12 illustratively communicates with the network 26 via blockchain exchange system 18, but in some embodiments, may optionally include communications controller 40 having processor, memory storage, and/or communication circuitry for communications with the network 26. Examples of suitable processors may include one or more microprocessors, integrated circuits, system-on-a-chips (SoC), among others. Examples of suitable memory storages, such as memory storage 36, may include primary storage and/or non-primary storage (e.g., secondary, tertiary, etc. storage); may include permanent, semi-permanent, and/or temporary storage; and/or may include memory storage devices including but not limited to hard drives (e.g., magnetic, solid state), optical discs (e.g., CD-ROM, DVD-ROM), RAM (e.g., DRAM, SRAM, DRDRAM), ROM (e.g., PROM, EPROM, EEPROM, Flash EEPROM), volatile, and/or non-volatile memory. Communication circuitry may include components for facilitating processor operations, for example, suitable components may include transmitters, receivers, modulators, demodulator, filters, modems, analog to digital converters, operational amplifiers, and/or integrated circuits.

The blockchain exchange system 18 communicates with a blockchain network 42 of medical device nodes 44. The nodes 44 are illustratively embodied as medical devices of the network which includes a blockchain exchange system 48, which have been authorized to participate in the blockchain ledger of medical device interactions. More particularly, the nodes 44 have been authorized to create new blocks for the blockchain.

Other medical devices 50 are illustratively authorized to communicate with the network 42, but do not include a blockchain exchange system 48. Still other medical devices 52 are illustratively authorized to communicate with the network 42, and include a blockchain exchange system 48, but have not been authorized to create blocks in the blockchain ledger of medical device interactions. Still other medical devices 54 are illustratively not authorized to communicate with the network 42 to access the blockchain ledger of medical device interactions. In the illustrative embodiment of FIG. 2, the semi-private topology is shown, although, the network 42 may be configured to have any suitable topology, including but without limitation, public, private, permissioned, and/or hybrid.

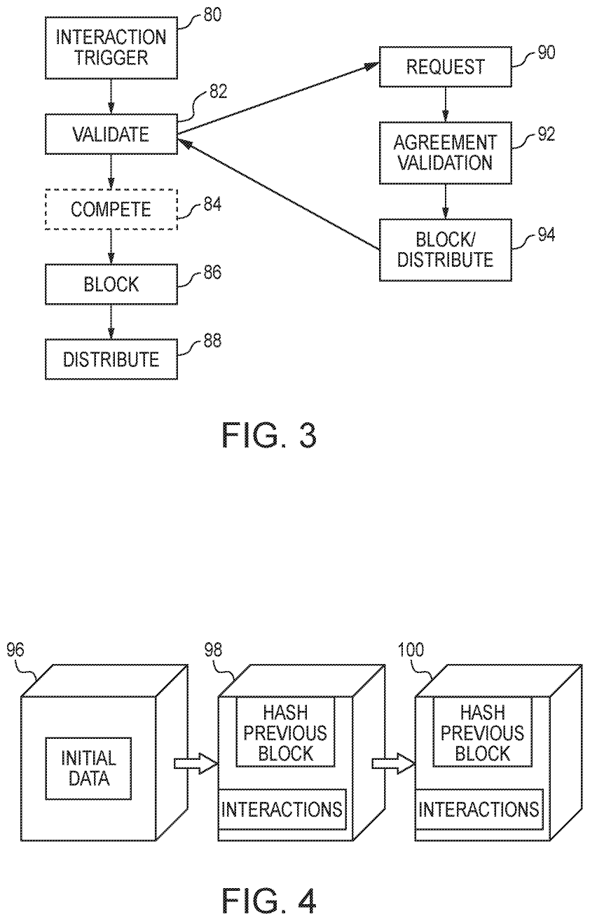

As shown in FIG. 3, a device interaction can be initiated by an interaction trigger 80. Responsive to the interaction trigger 80, a validation 82 can be performed to determine whether medical devices are authorized to access the blockchain ledger of medical device interactions. Responsive to validation 82, an optional competition 84 can be conducted to determine the medical device to generate the next block in the chain. Responsive to validation 82 and competition 84 (if applicable), a block can be formed 86 recording the medical device interaction(s). Responsive to block formation 86, the new block can be distributed 88 throughout the network 42 for ratification.

The interaction trigger 80 illustratively includes the threshold operation which constitutes medical device interaction. Continuing from the earlier example of medical device proximity, the interaction trigger 80 includes medical devices coming within a threshold distance of each other. For example, the IV pump 22 is brought within the room of the patient bed 12 and is placed within 5 feet of the patient bed 12, which is predetermined as the threshold interaction proximity. As previously mentioned, other medical device interactions may include direct and/or indirect communications and/or physical and/or RF connections between the medical devices; and/or application of the medical devices to common patients, by common caregivers, and/or in common procedure types. Corresponding interaction triggers 80 may include initial communication connections, application prompted search for similarly applied medical devices, and/or periodic checks for medical device application.

The validation 82 illustratively includes communication of identification regarding the medical devices of the interaction. Continuing from the earlier example of the IV pump 22 being placed within the proximity threshold of the patient bed 12, the patient bed 12 illustratively receives identification information of the IV pump 22. The identification information includes can include any one or more of a serial number, model number, device type, manufacturer, facility-assigned ID, location, communication certificate, root/intermediate certificate, secure key for blockchain exchanges, and/or other information identifying the medical device to the patient bed 12.

In the example, the blockchain ledger does not require the IV pump 22 to be authorized as a part of the blockchain network 42 in order to record the interaction as valid. Thus, the blockchain exchange system 18 of the patient bed 12 is configured for recording interaction with the IV pump 22, regardless of whether the IV pump 22 is authorized as part of the blockchain network 42, and the validation 82 can be completed merely by communication of identification information.

However, in some embodiments, the blockchain ledger may be configurable to restrict valid interactions to those interactions between blockchain authorized medical devices. Continuing the example of the patient bed 12 and the IV pump 22, the validation 82 may require that the IV pump 22 provide a blockchain authorization which can include presentation of a communication certificate, root/intermediate certificate, secure key for blockchain exchanges, and/or other information authorizing blockchain activity, for example, authorization for communication with the blockchain network 42. Because the blockchain authorization can be managed at the device level by local storage of the blockchain authorization information, for example, stored on the IV pump 22 itself, the need for remote access can be reduced including the need of frequent remote access for each medical device interaction with a blockchain authorized medical device.

According to the configuration of the blockchain ledger, if an interaction trigger 80 occurs with a medical device lacking blockchain authorization, an agreement validation can be required. Continuing from the example of the patient bed 12 and the IV pump 22, the IV pump 22 comes into threshold proximity with the patient bed 12 achieving the interaction trigger 80 and activating the validation 82. The validation 82 requires agreement validation because the IV pump 22 does not have blockchain authorization. Accordingly, a request 90 is sent to the network 26. By example, the patient bed 12 sends a request for authorization including identification information of the other medical device, e.g., the IV pump 22.

Upon successful request, an agreement validation 92 can be performed. In the illustrative embodiment, the agreement validation 92 is illustratively conducted within a data marketplace layer for management and access to blockchain data. The server 28 illustratively performs operations of the agreement validation 92 based on the identification information. The agreement validation 92 illustratively includes accessing an agreement profile for the medical device interaction data conditions. The agreement profile can be customized according to care facility, medical device type, device location, data access type, quality of service (including frequency, duration, priority, and/or rate of data access), and/or any other suitable parameters. The server 28 at item 92 builds and executes the agreement based on the agreement profile, as a smart contract, using a blockchain intelligent signature. The completed agreement provides a communication key, illustratively embodied as a secure communication key, for communication between medical devices, although in some embodiments, the communication key may include a communication certificate, root/intermediate certificate, and/or other suitable manner of allocation element.

Upon successful agreement validation, a block can be created to record the validation 94. In the illustrative embodiment, the block creation 94 includes formation of the agreement validation transaction as a block in a blockchain agreement ledger. The blockchain agreement ledger is illustratively embodied as a distributed ledger, similar to the device interaction ledger, but distributed and stored at the server-level. Upon successful block creation, an indication of agreement validation can be communicated to the medical devices (returning to block 82). Upon successful validation in block 82, the process can continue.

Upon successful validation in block 82, an optional competition 84 can be performed. The competition 84 illustratively includes the blockchain consensus operation algorithm. The consensus operation is illustratively embodied as a proof-of-work operations in which participating medical device nodes compete to solve the consensus puzzle (non-trivial task) with the winner forming and distributing the next block in the chain. In some embodiments, the consensus operation may include any of proof-of-capacity, proof-of-stake, proof-of-authority, Byzantine fault tolerance, and/or any other suitable manner of consensus.

The successful resource creates a new block 86 to record the medical device interaction. The new block is distributed 88 to the relevant nodes to complete the recordation. Accordingly, the blockchain medical device interaction ledger can be maintained.

In the principle example, the patient bed 12 illustrative conducts the operations discussed above regarding block 82 and the optional competition determines which medical device node of the network 42 would perform block creation 86 and/or distribution 88. In the illustratively embodiment, the patient bed 12 performs the communications with the network 26, but in some embodiments, either of the medical devices of the interaction may communicate with the network 26, whether directly or indirectly. The distributed nature of the processing resources takes advantage of the shared dynamic of processing power across multiple medical devices, allowing the individual resources of a given medical device, such as the patient bed 12 to be reduced. This reduced need for resources can likewise translation to reductions in auxiliary equipment such as cooling devices and power equipment.

Referring now to FIG. 4, an illustration of the blockchain configuration is shown. Initial data is illustratively stored as a genesis block 96 that is hard-coded into the blockchain. As the blockchain adds additional linked blocks to the chain, it forms a distributed linked list of records. A subsequent block 98 is formed including a hash of the genesis block 96 as the previous block in the chain. A further block 100 is formed including a hash of the block 98, and so forth. The contents of each block are therefore digitally signed under the cryptographic hashing, which defends against manipulation of the individual blocks in the chain.

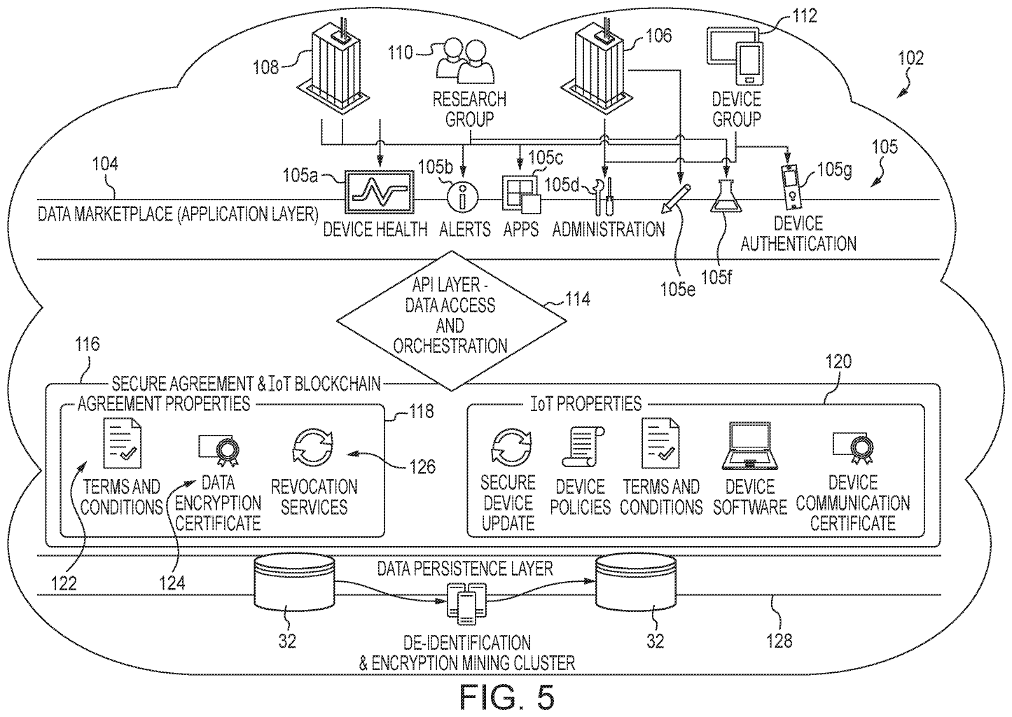

Referring now to FIG. 5, an exemplary application arrangement 102 is shown for implementation of the medical device interaction and/or agreement blockchains. A Blockchain Smart IoT & Data Marketplace layer 104 illustratively provides an interface layer for secure sharing and exchange of data via the blockchain agreements. The Smart IoT & Data Marketplace layer 104 can interface with a principle administrator 106 for governing blockchain operation, third party administrators 108 for interaction with blockchain operations, third party research groups 110 having customized access to blockchain operations, and/or individual devices 112 for administration and/or access to blockchain operations. The Smart IoT & Data Marketplace layer 104 may also provide access to various aspects of the medical device operations 105 includes device health 105a, alerts 105b, applications 105c, administrative tools 105d, editing tools 105e, therapeutics 105f, and/or device authentication operations 105g.

The Blockchain Smart IoT & Data Marketplace layer 104 is connected with sub-layers via an application program interface (API) layer 114 for data access and orchestration. The API 114 provides access to secure agreement and IoT blockchain properties 116 including agreement properties 118 and/or IoT properties 120. The agreement properties 118 can include terms and conditions 122, encryption certificates 124, and/or revocation conditions 126. The IoT properties 120 can include secure device update information, device policies, terms and conditions, device software, and/or communication certificates.

A data persistence layer 128 can be applied to perform data de-identification to protect against dissemination of personal healthcare information, and/or encryption mining operations to mine information according to the usage needs. For example, although the blockchain device interaction data may include merely “process of care” information that is information having no patient-related identification, should any cross-correlation between data sets permit undesirable personal health information (PHI) to be discovered, de-identification may be performed to avoid sharing of such information, for example, through anonymization among other approaches. Such de-identification can be used to comply, or rather avoid the need for compliance, with HIPAA privacy rules.

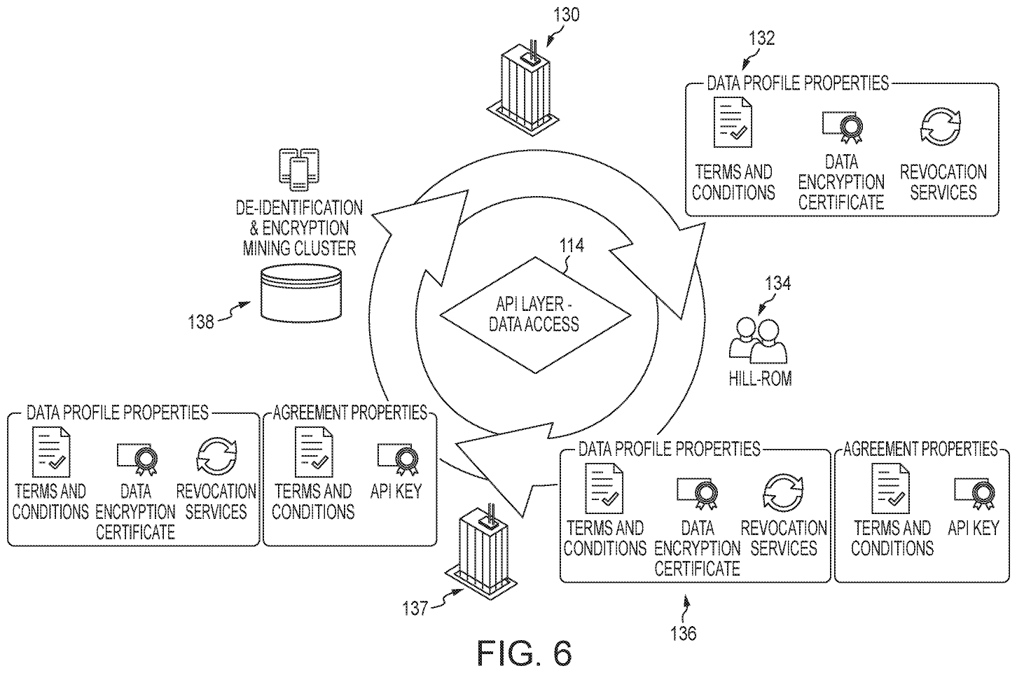

Referring to FIG. 6, an exemplary process of a Blockchain Smart IoT & Data Marketplace is shown at the institutional level. At 130, an organization can request access to medical device interaction blockchain data. The request may be for commercial and/or research-level access, and access may be tailored accordingly. At 132, the blockchain agreement validation builds and executes the agreement. In the illustrative embodiment, the blockchain agreement validation is executed on the server 28, which may be cloud-based, and the agreement profile can include terms and conditions, root communications certificate, and revocation services.

At 134, the agreement can be approved by the principle administrator, e.g., Hill-Rom Services Inc., and is intelligently signed by blockchain. At 136, an API key is generated and communicated to the buyer/subscriber 137 indicating an approved smart contract for data access among data profile properties and/or agreement properties. At 138, the API key and requestor identification secret is applied to authenticate an API request. The API 114 sends the request to the data mining layer, and the request can indicate the data profile properties and/or agreement properties. At 138, de-identification and/or mining operations can be conducted to assemble usable data. De-identification and/or mining operations can be performed at the server-level, e.g., by server 28, using the medical device interaction ledger data. Mined and/or de-identified data can be provided to the organization at 130.

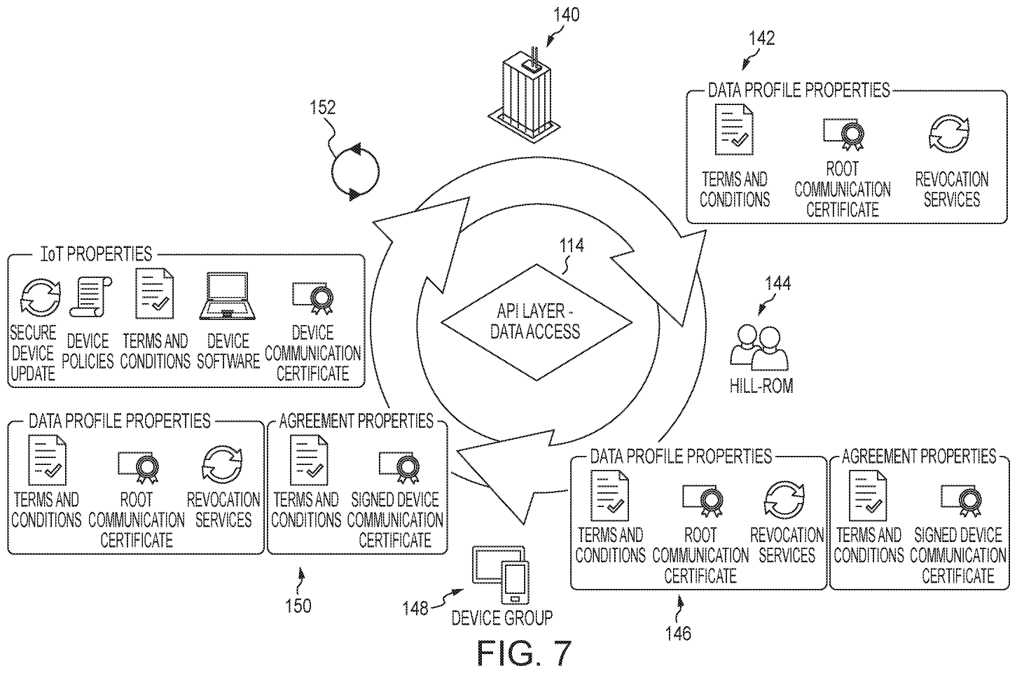

Referring to FIG. 7, an exemplary process of Blockchain Smart IoT & Data Marketplace is shown at the partner level. A potential partner platform 140 can request access for medical device interaction blockchain data. The request can be embodied as an individual medical device interacting with a node 44 of the medical device network 42 and initiating a request, and/or can include a family-based request for authorization of medical devices from a particular group, such as a particular manufacturer. At 142, the blockchain agreement validation builds and executes the agreement. In the illustrative embodiment, the blockchain agreement validation is executed on the server 28, which may be cloud-based, and the agreement profile can include terms and conditions, root communications certificate, and revocation services.

At 144, the agreement can be approved by the principle administrator, e.g., Hill-Rom Services Inc., and is intelligently signed by blockchain. At 146, a device communication certificate (and/or key) is generated and communicated to the requesting medical device (and/or group) 148 indicating an approved smart contract for data access among data profile properties and/or agreement properties. At 148, the API key and requestor identification secret is applied to authenticate an API request. At 150, the blockchain medical device interaction exchange can occur, including identifying authorized medical devices, and can indicate the IOT properties, data profile properties, and/or agreement properties. Newly authorized medical devices are accepted into the blockchain. At 152, information can be exchanged as permitted between the blockchain medical device network 42 and the requesting entity. For example, contractual obligations (such as payments) can be effected outside the blockchain device interaction network based on the blockchain device interaction data.

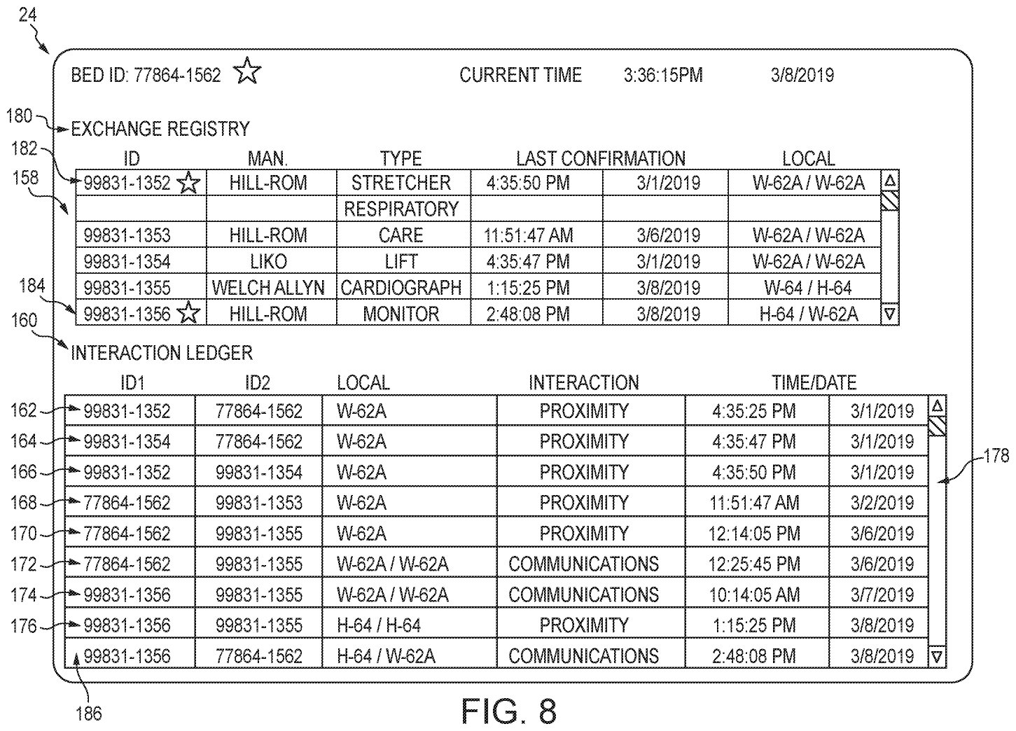

Referring now to FIG. 8, a screenshot of a display 158 provided by the graphical user interface 24 of the patient bed 12. The GUI 24 illustratively provides user access to the medical device interaction blockchain ledger 160. The ledger 160 is shown as a list of medical device interaction events recorded on the medical device interaction blockchain.

The ledger 60 illustratively indicates the medical devices involved in the recorded interaction, the location, the type of interaction, and the time and date of interaction. For example, in row 162, the identification code of a stretcher is indicated as identification number 99831-1352 having interacted with the patient bed 12 indicated by identification number 77864-1562, at room W-62A within the care facility, based on proximity between the medical devices, at 4:35:25 PM on Mar. 1, 2019. Other interactions include: at row 164, the patient bed 12 interacted with the lift 20; at row 166, the lift 20 and stretcher interacted; at row 168, the patient bed 12 interacted with a respiratory care device, such as a nebulizer, indicated by identification number 99831-1353; at row 170, the patient bed 12 interacted with a cardiograph indicated by identification number 99831-1355 by proximity; at row 172, the cardiograph interacted with the patient bed 12 again, but by communications with the patient bed 12; at row 174, the cardiograph interacted with a monitor indicated by identification number 99831-1356 by communication with the monitor; at row 176, the monitor interacted with the cardiograph, by proximity, but in location H-64 of the care facility, which corresponds to a hallway in which the two medical devices were located. In row 186, the monitor in the hall H-64 interacted with patient bed 12 while in the room W-62A by wireless communication.

The interaction ledger 60 illustratively includes a scroll bar 178 for scrolling through the ledger entries. The headings, ID1, ID2, LOCAL, INTERACTION, TIME/DATE are illustratively embodied as sortable fields that can be selected by the user, for example, by contact in touchscreen implementations of the GUI 24. Accordingly, the user can access and interact with the medical device interaction blockchain data via the GUI 24.

The display 158 of the GUI 24 illustratively displays an exchange registry 180. The exchange registry 180 indicates the medical devices that have been authorized for access to the medical device interaction blockchain data. The exchange registry 180 illustratively indicates the identification number for the medical device, the manufacturer, device type, time/date of last known confirmation action, and location information. For example, at row 182, the stretcher is indicated as authorized for access to the medical device interaction blockchain data. The stretcher is identified by identification number 99831-1352; is manufactured by Hill-Rom Services, Inc.; is a stretcher type of medical device; was last confirmed as authorized for access with the network 42 on Mar. 2, 2019 at 4:35:25 PM at location W-62A of the care facility.

The date/time of last known confirmation for each medical device is illustratively determined indirectly as the latest authorized record entry of each medical device onto the interaction ledger 160. For example, in comparison with the interaction ledger 160, the exchange registry 180 indicates at row 182 that the stretcher was last confirmed as authorized on Mar. 2, 2019 at 4:35:25 PM, and the interaction ledger 160 indicates a proximity interaction between the patient bed 12 and the stretcher at the same time. However, in some embodiments, the medical device blockchain exchange network 42 may periodically query the Blockchain Smart IoT & Data Marketplace, for example, by communication with the server 28, to confirm authorized medical devices on the exchange registry 180.

As shown in FIG. 8, at the title block in row 184, the patient bed 12 is indicated with its identification number 77864-1562 which is indicated by a star as a node 44 of the network 42 of medical devices. In the illustrative embodiment, the status of the patient bed 12 as a node 44 is indicated by a star, although in some embodiments, any suitable indication of the GUI 24 may be applied.

Notably, among the other medical devices on the exchange registry 180, only the stretcher (no. 99831-1352) and the monitor (no. 99831-1356) are nodes 44 of the network 42 of medical devices as indicated by a star in their listing on the exchange registry 180. Thus, among the list of medical devices in the exchange registry 180, only the patient bed 12, the stretcher, and the monitor are nodes 44 of the network 42, and the other medical devices merely communicate with the network 42 and may or may not be authorized for access to the medical device interaction blockchain data. As previously mentioned, as a node 44 of the medical device blockchain network 42, the patient bed 12, stretcher, and monitor each illustratively include a blockchain exchange system 18 and can compete to form new blocks and to maintain the distributed ledger. Accordingly, the processing resources of the patient bed 12, stretcher, and monitor can be pooled to address the medical device interaction needs. Such resource pooling can reduce the need for increased processing power in any particular medical device to enable the blockchain ledger for medical device interactions.

As shown in FIG. 8, the location column entitled LOCAL provides an indication of the location at which a last known confirmation of the medical device has occurred. For example, at row 182, the stretcher was confirmed at room W-62A by interaction with another device at room W-62A, and the location of the stretcher and other device is shown as W-62A/W-62A. Again, comparing the registry 180 with the ledger 160 at row 162, it can be understood that the other medical device was the patient bed 12, in this instance, and each of the patient bed 12 and the stretcher were located at the room W-62A. Further, at row 184, the monitor (no. 99831-1354) was last confirmed at location H-64 by interaction with a medical device at W-62A, and in reference to the ledger 160 at row 186, that medical device was the patient bed 12 within room W-62A of the care facility and the type of interaction was communication between the monitor and the patient bed 12, remotely, between the hall and the room.

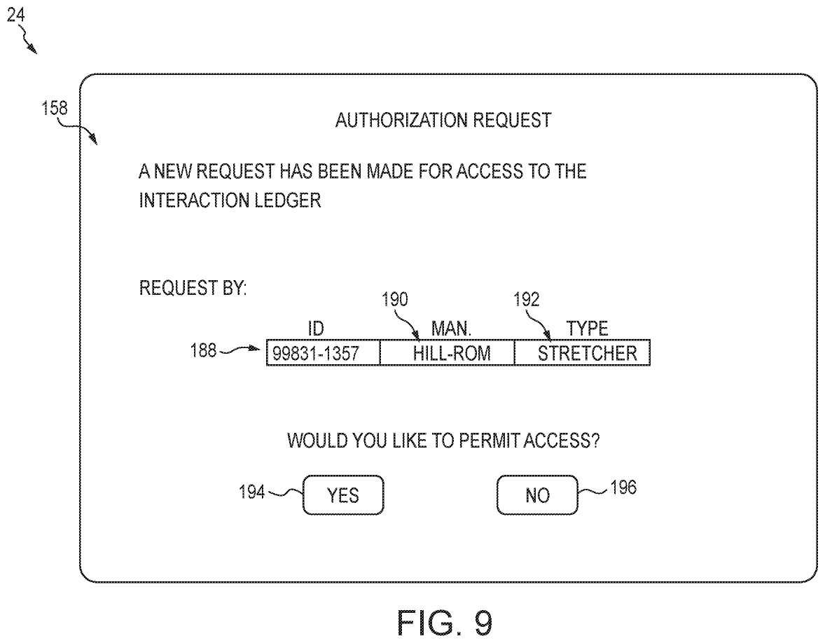

Referring now to FIG. 9, an authorization request screen is shown on the display 158 of the GUI 24. The authorization request indicates that another stretcher, having identification no. 99831-1357, has interacted with the patient bed 12 and is requested access to the medical device interaction data on the blockchain ledger. In some embodiments, the interaction may have occurred between the new stretcher and any medical device connected with the medical device network 42, and the request authorization is displayed on the GUI 24.

The request includes the medical device identification number 188, the manufacturer information 190, and the medical device type 192. Confirmation buttons 194, 196 can be selected by the user to allow or deny the authorization request. In the illustrative embodiment, the authorization request is implemented in addition to and parallel with the authorization request at the server level, however, in some embodiments, the device level authorization request may be implemented as condition precedent to the request at the server level, and/or can be implemented in place of the server level request.

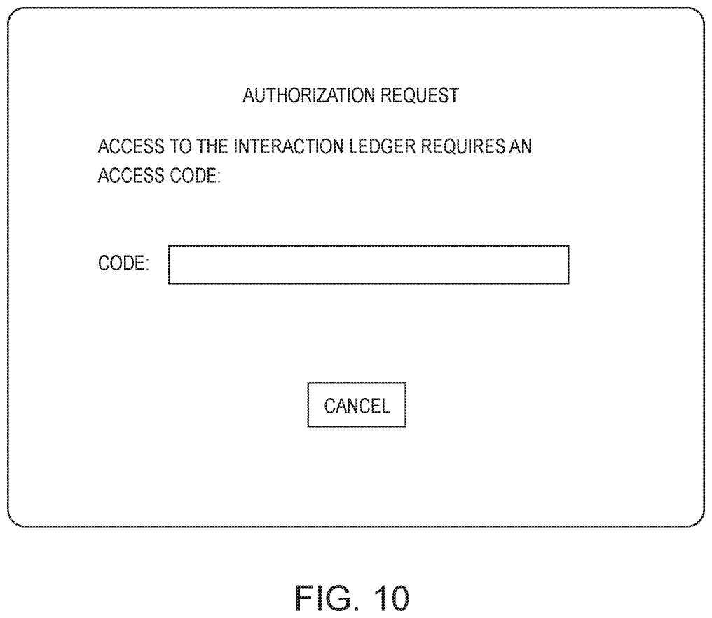

As shown in FIG. 10, upon user selection of the “Yes” button 194 to confirm authorization of the device request for access to the medical device interaction data on the blockchain ledger, an access code can be required. The screen 158 illustrative shows a prompt 198 requesting entry of the access code. The access code is illustratively obtained from the server 28 upon agreement validation, and can be entered by the user, by selecting the code entry field 200 and inputting the code via input device such as a keyboard, or via electronic keyboard available on the GUI 24. In the illustrative embodiment, entry access code is implemented in addition to and parallel with the authorization request at the server level, however, in some embodiments, the device level entry of the access code may be implemented in place of the server level indication of the communication key. A cancellation button 202 is presented for user selection to terminate the authorization.

Medical device information management using blockchain can offer an opportunity for infrastructure for the data between devices and process. Technology within the present disclosure can enable care facilities with rich signal only information to automate workflows and automatically enable smart type contracts which can develop a new marketplace for healthcare data. Moreover, blockchain transactions need processing power to approve each transaction of data. There are often a large number of processors in a care facility, such as a hospital, comprised within common medical devices.

Within the present disclosure, the same device that provides care, now can be used to discern more information about care with data from other products while providing an alternate pathway to generate services through a data marketplace. For instance, medical product providers which provide medical devices to a hospital at a special rate with the understanding that certain protocols will be followed. For example, a patient bed provider, such as Hill-Rom Services, Inc., may provide patient beds to a care facility with the expectation that patient rotation therapies will be undertaken to reduce pressure-related injuries. The providers desires to ensure that protocols of turning a patient and progressive mobility are being followed. Blockchain medical device interaction data can be appended with this information to allow monitoring of whether protocols were followed, or alternatively, the bed operation data (rotation therapy information) could be made accessible with the device interaction data. Parties to a contract could be informed of the medical device interaction data and/or rotation therapy status of the beds, and whether contract terms would be cancelled or amended.

Within the present disclosure, blockchain communications can also offer the unique capability to transfer transactions from hospital to hospital without a true EMR. Hospitals could develop contracts that share the minimum dataset of patients with each other for care purposes. Blockchain device interaction data of the present disclosure can provide and/or track the equipment that was used on patients between care providers as a consideration of infection control, for example, for issues of MRSA and C. DIFF, blockchain could have a record of each device and patient/staff (via identification badges as medical devices) that interact. The medical device network is formed by the devices and workflows already present; utilizing processing power of the devices and cloud infrastructure to truly change the care delivery ecosystem.

Within the present disclosure, a blockchain smart IoT & data marketplace built on blockchain and big data technology, can provide solutions for implementing and accessing a record of medical device and/or patient interactions from birth to death with each participant (caregive to medical device) also having its own record from “birth” of the experience. Implementations of within the present disclosure include a linked network of interaction points between patients, physicians, nurses, external entities, and medical devices. For example, medical devices communicating to record interactions can be paired with patient's through there assigned patient beds, and caregivers can be paired with identification badges (as medical devices) and/or through the beds and/or patients themselves. The devices can be mapped in a secure and immutable manner.

Blockchain smart IoT & data marketplaces within the present disclosure can provides a foundation to support better care through the entire life of a patient, enhance early prevention statistics, build trust in data for care & research purposes, intelligently manage data sharing agreements, secure smart IoT devices, provide insight into the real-world usage of medical devices and/or build an economy of medical data down to the sensor level. At the foundation, a smart IoT and data marketplace can provide: a layer to securely share and purchase data via blockchain immutable contracts of ownerships and rights for which data can added, shared and sold under these contracts; encrypted storage of data linked to smart contracts; a device blockchain for secure IoT; a data marketplace; and/or big data storage platforms. Such a marketplace can provide interfacing with smart contract profiles for data sharing control; definition of rights/properties for which owner data can added, anonymized, shared, and/or exchanged; interfacing for data sharing requests; data access requests can initiate a secure contract between the owner and subscriber (see contract layer); owners can revoke the contract at any point with the contract holding the same legal binding found in existing data sharing contracts; operation can be leveraged as part of a device subscription model or existing pricing model; data to the sensor level for IoT devices and data point level for data storage layers; subscription model in which subscribing to the feed from a sensor will process micropayments, or accumulate like a utility company; data can be leveraged by analytical companies looking for build analytical solutions on connected devices within a care facility; data can be leverage by research organizations for patient outcomes; data an be leveraged for competitor access for data coming from systems owner; for example, competitors can subscribe to specific data points/sensors based on the contractual layer agreement to allow cross-platform collaboration; device data can be configured for limitations on data type, such as merely process-of-care, unidentified data, and/or “identifiable” patient or EMR data following HIPPA and GDPR guidelines as required. Secure contract layers can be leveraged for rights and ownership of data. Owners can choose to exchange data as they please under associated guidelines but operations can be intelligently managed via smart contract. For “anonymized” data (patient, EMR, diagnosis, usage . . . etc.) “miners” can be used to anonymize the data and certify for marketplace sharing. Secure contract layer can define ownership and rights to the anonymized data. Anonymized data can be valuable for research and development, sales and/or other opportunities, and can be available via subscription and/or one-time purchase.

Within the present disclosure, a blockchain agreement layer can maintain validated owner and buyer identification; maintain smart contract agreements in a blockchain. Contractual agreements can be built on compliance and legal requirements. Smart contracts can include the hashed link to the data security encryption & API key. The API key and encryption certificate can be required for blockchain data access, can contractually identify and/or document at the data level, ownership and rights for which data can added, shared and sold under these contracts. The API key and encryption certificate can enable the contractual process for selling of anonymized data between network provider, data owners and researchers, and/or can enable contractual process for sharing of data between healthcare organizations for patient care purposes. The blockchain agreement layer can be driven by an owner, such as Hill-Rom Services, Inc., and a partner IoT for benchmarking and care. Healthcare organizations may elected to opt out of benchmarking with their data. The blockchain agreement layer can preform required to continuously update recommendations and diagnosis procedures for smart contract in realtime for different solutions including machine learning scenarios.

Within the present disclosure patients can review their lifeline of data which will provide indicators based on analytics and health recommendations. Additionally, these indicators can be real-time updates via machine learning. Patients can contractually opt-out where applicable for full control at the patient ownership level. Contractual sharing of data between competitive implementations of devices. In disclosed implementations, a record of all medical devices can include, without limitation: configuration, Serial Number/Identification, Basic information (model type, version, software/firmware), Location, Communication Certificate, basic communication (validation of blockchain device communication), initial device registration based on root/intermediate certificate signature, toot/intermediate certificate storing in blockchain, enhancements to IoT security as blockchain is native to security as participants in a blockchain must verify a transaction before it is accepted as legitimate, interactions conceptually can be considered as device authentication or any communication, interactions can be inked to secure contract layer for intelligent management of data point and sensor level subscriptions.

Within the present disclosure, blockchain smart IoT & data marketplace workflows can include request being initiated in Data Marketplace for access to anonymized (Navicare) data, blockchain validation of the contractual profile of data and building of contractual agreement, agreement approval and signing intelligently by blockchain, API key can be the output of an approved smart contract for data access and provided to the “buyer/requester,” API key and requestor identification secret key can be used to authenticate an API request, API can send requests to data mining layer, and data mining layer can processes smart contract blockchain to confirm data access and rights. If blockchain identification is successful, data can be decrypted and transmitted securely through API layer to consumer. The throughput of API calls are monitored and, if payment agreement is in place, transactions and amounts can be documented in contractual blockchain for processing.

Within the present disclosure the workflow IoT can initiate request in Data Marketplace for access to medical device data to flow through a NurseCall system in the same healthcare facility, blockchain operation can validate the agreement profile of medical device data for that location and can build contractual agreement. The agreement can be approved by the owner and signed intelligently by blockchain. A device communication key can be output from an approved smart contract for data access and provided to the “buyer/requester.” The Nursecall may be required to register with IoT blockchain as a participant and may send “registration” request to IoT blockchain via API layer. The IoT processes smart contract blockchain to confirm identity of Nursecall, data access and rights to owner devices. If blockchain identification and authorization is successful, the Nursecall can now a participant in the IoT blockchain. The Nursecall can send requests as IoT participant to owner devices, or data can be pushed to the Nursecall (depending on IoT architecture). Source and/or destination devices can be always authenticated via the IoT blockchain. Data can be transmitted securely to the consuming device. Throughput can be monitored and if payment agreement is in place, transactions and amounts can be documented in contractual blockchain for processing.

Within the present disclosure medical device blockchain operation is a virtually incorruptible digital ledger of medical device transactions. By allowing medical device digital information to be distributed but not copied, blockchain technology created the backbone of a new type of internet. The medical device blockchain networks within the present disclosure, can live in a state of consensus, one that automatically checks in with itself every ten minutes. A kind of self-auditing ecosystem of a digital value, the network reconciles every transaction that happens in ten-minute intervals. Each group of these medical device transactions is referred to as a “block.” Two important properties result from this: transparency data is embedded within the medical device network as a whole, by definition it is public, and it cannot be corrupted altering any unit of information on the medical device blockchain would mean using a huge amount of computing power to override the entire network.

Although certain illustrative embodiments have been described in detail above, variations and modifications exist within the scope and spirit of this disclosure as described and as defined in the following claims.

We claim:

1. A medical device communication system for blockchain exchange, the system comprising:
a patient bed for supporting a patient above the floor, the patient bed including a blockchain exchange system including a processor executing instructions stored on at least one memory storage and communication circuitry for communication with other medical devices,
a remote server arranged in communication with the patient bed, and
a network of medical device nodes each including a blockchain exchange system including a processor executing instructions stored on at least one memory storage and communication circuitry for communication with other medical device nodes of the network,
wherein the blockchain exchange system of at least one of the patient bed and one of the medical device nodes is configured to validate interactions between the patient bed and other medical devices, and each of the blockchain exchange systems is configured to record valid interactions linked with at least one previous valid interaction between the patient bed and other medical devices.

2. The medical device communication system of claim 1, wherein the blockchain exchange system of the patient bed validates interactions between the patient bed and other medical devices.

3. The medical device communication system of claim 1, wherein the blockchain exchange systems of the patient bed and at least one of the medical device nodes compete to determine which will validate an interaction between the patient bed and another medical device.

4. The medical device communication system of claim 3, wherein the successful one of the blockchain exchange systems of the patient bed and at least one of the medical device nodes competing to determine which will validate an interaction between the patient bed and another medical device, sends a validation signal indicating the valid interaction to the other medical device nodes of the network.

5. The medical device communication system of claim 1, wherein the each of the blockchain exchange systems of the patient bed and the medical device nodes maintains an identical ledger of valid interactions.

6. The medical device communication system of claim 1, wherein the interactions between the patient bed and other medical devices includes an exchange of information between the patient bed and the other medical device.

7. The medical device communication system of claim 6, wherein the exchange of information is process-of-care information.

8. The medical device communication system of claim 6, wherein the exchange of information does not include Protected Health Information.

9. The medical device communication system of claim 1, wherein the remote server is configured to perform agreement validation to permit a medical device to join the network of medical device nodes.

10. The medical device communication system of claim 9, wherein the agreement validation is a blockchain exchange.

11. The medical device communication system of claim 10, wherein the agreement validation includes smart contract formation hosted on the remote server.

12. The medical device communication system of claim 11, wherein smart contract formation includes authorization for access to recorded valid interactions between patient bed and other medical devices.

13. The medical device communication system of claim 12, wherein the recorded valid interactions are encrypted.

14. A medical device communication system for blockchain exchange, the system comprising:
a patient bed for supporting a patient above the floor, the patient bed including a blockchain exchange system including a processor executing instructions stored on at least one memory storage and communication circuitry for communication with other medical devices, wherein the blockchain exchange system is configured to validate and record interactions between the patient bed and other medical devices, wherein each valid interaction is linked with at least one previous valid interaction,
a remote server arranged in communication with the patient bed, and
a network of medical devices each including a processor executing instructions stored on at least one memory storage and communication circuitry for communication with other medical devices of the network, wherein at least one of the medical devices of the network forms a network node configured to validate and record interactions between the patient bed and other medical devices.

15. The medical device communication system of claim 14, wherein the patient bed and at least one of the medical devices forming the network node compete to determine which will validate an interaction between the patient bed and another medical device.

16. The medical device communication system of claim 15, wherein the successful one of the patient bed and at least one of the medical device forming the network node competing to determine which will validate an interaction between the patient bed and another medical device, sends a validation signal indicating the valid interaction to the other medical devices of the network.

17. The medical device communication system of claim 14, wherein each of the patient bed and the medical devices of the network maintains an identical ledger of valid interactions.

18. The medical device communication system of claim 14, wherein the interactions between the patient bed and other medical devices includes an exchange of information between the patient bed and the other medical device.

19. The medical device communication system of claim 18, wherein the exchange of information is process-of-care information.

20. The medical device communication system of claim 18, wherein the exchange of information does not include Protected Health Information.

21. The medical device communication system of claim 14, wherein the remote server is configured to perform agreement validation to permit a medical device to join the network of medical devices.

22. The medical device communication system of claim 21, wherein the agreement validation is a blockchain exchange.

23. The medical device communication system of claim 22, wherein the agreement validation includes smart contract formation hosted on the remote server.

24. The medical device communication system of claim 23, wherein smart contract formation includes authorization for access to recorded valid interactions between patient bed and other medical devices.

25. The medical device communication system of claim 14, wherein the recorded valid interactions are encrypted.
